(19) United States
(12) Patent Application Publication
(10) Pub. No.: US 2018/0360405 A1
(43) Pub. Date: Dec. 20, 2018

(54) CT PERFUSION PROTOCOL TARGETING

(71) Applicant: KONINKLIJKE PHILIPS N.V., EINDHOVEN (NL)

(72) Inventors: Sven PREVRHAL, HAMBURG (DE); Holger SCHMITT, LUETJENSEE (DE); Hannes NICKISCH, HAMBURG (DE)

(73) Assignee: KONINKLIJKE PHILIPS N.V., EINDHOVEN (NL)

(21) Appl. No.: 16/060,473

(22) PCT Filed: Dec. 20, 2016
(86) PCT No.: PCT/EP2016/081904

§ 371 Date: Jun. 8, 2018

Related U.S. Application Data
(60) Provisional application No. 62/270,847, filed on Dec. 22, 2015.

(51) Int. Cl. A61B 6/00; A61B 6/03

(52) U.S. Cl. CPC A61B 6/507; A61B 6/486; A61B 6/032; A61B 6/5205; A61B 6/503; A61B 6/54

(57) ABSTRACT

A system (100) for a targeted perfusion scan includes a computed tomography (CT) scanner (120), a feeding territory map (132) and a targeted perfusion unit (140). The CT scanner (120) performs a perfusion scan of a portion of tissues of an organ. The feeding territory map (132) maps arterial locations of an arterial vessel tree to spatially located organ tissues of the organ fed by the arterial locations. The targeted perfusion unit (140) includes one or more processors (164) configured to determine targeted coverage (200) from a location of a stenosis (112, 210) and the feeding territory map, and to control the CT scanner to perform the perfusion scan according to the determined targeted coverage.

### FIELD OF THE INVENTION

The following generally relates to medical imaging with specific application to computed tomography (CT) perfusion scanning, such as perfusion scanning of organs, such as a heart, a liver or a brain.

### BACKGROUND OF THE INVENTION

A CT perfusion protocol uses a CT scanning device to scan the myocardium of a heart during uptake of an administered contrast agent to identify and diagnose myocardial defects. During the perfusion scan a CT rotating gantry scans at a same axial position or z-axis with a portion of the heart in the field of view to observe the rise of contrast in the tissues. An X-ray source emits x-rays that traverse the tissues and are detected by a radiation detector. The detected x-rays are reconstructed into CT images that are then fused to a perfusion image. The same axial position corresponds to a position along a longitudinal axis of the patient, e.g. patent remains in the same position relative to the plane of the rotating gantry. The rise of contrast in the myocardial tissues occurs in seconds. Missing the uptake period can mean waiting for washout and re-administering the contrast agent, and exposing a patient to additional ionizing radiation from the CT scanning device. The CT scanning device is typically configured to monitor for the contrast agent with a lower dose scan before switching to a higher dose with higher resolution during the uptake.

Typical CT scanning devices have a field of view sufficient only to scan a portion of the heart. One approach to ensuring the scanning potentially damaged myocardium is to manufacture the CT scanning device with a large field of view or scan coverage sufficient to repeatedly scan the entire heart at the same axial position. With the entire heart being scanned in each rotation, the uptake in all of the myocardium can be observed. However, this also means that a larger portion of tissues including the entire heart are subjected to a larger dose of ionizing radiation during monitoring and scanning, and ionizing radiation is an accumulated dose for the patient. Another approach is to use a shuttle mode where a patient in moved in a limited field of view at incremental positions, e.g. back and forth between two positions, to expand the scan coverage. However, the shuttle mode increases the dose and can miss the peak enhancement during uptake.

Myocardial defects, such as damaged heart muscle, are typically caused by lack of oxygen to the myocardial tissues attributable to blockages in the arterial supply to the myocardial tissues, e.g. atherosclerotic coronary artery disease (CAD).

### SUMMARY OF THE INVENTION

Aspects described herein address the above-referenced problems and others.

The following describes a method and system for targeting the CT perfusion scan coverage of a portion of an organ, such as the heart, the liver or the brain. The portion of the organ imaged during the perfusion scan is identified according to spatially located stenosis and a feeding territory map of the organ tissues. In one embodiment, the stenosis can include a stent location. In one embodiment, the stenosis is spatially located using a CT angiography scan.

In one aspect, a system for a targeted perfusion scan includes a computed tomography (CT) scanner, a feeding territory map and a targeted perfusion unit. The CT scanner performs a perfusion scan of a portion of tissues of an organ. The feeding territory map maps arterial locations of an arterial vessel tree to spatially located organ tissues of the organ fed by the arterial locations. The targeted perfusion unit includes one or more processors configured to determine targeted coverage from a location of a stenosis and the feeding territory map, and to control the CT scanner to perform the perfusion scan according to the determined targeted coverage.

In another aspect, a method of targeting a perfusion scan includes determining targeted coverage from a location of a stenosis and a feeding territory map. A computed tomography (CT) scanner is controlled to scan a portion of organ tissues according to the targeted coverage during the perfusion scan.

In another aspect, a non-transitory computer readable storage medium contains instructions that when executed by one or more processors determine a targeted coverage of a computed tomography (CT) scanner from a location of a stenosis in an arterial vessel tree of an organ and a feeding territory map that maps arterial locations of the arterial vessel tree to spatially located organ tissues of the organ fed by the arterial locations. The instructions further control the CT scanner to perform a targeted CT perfusion (CTP) scan according to the targeted coverage.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an embodiment of a targeted perfusion protocol system.

FIG. 2 illustrates an exemplary heart scan targeted coverage with a stenosis and corresponding perfusion defect.

FIG. 3 flowcharts an embodiment of a method of targeting perfusion scanning coverage.

### DETAILED DESCRIPTION OF EMBODIMENTS

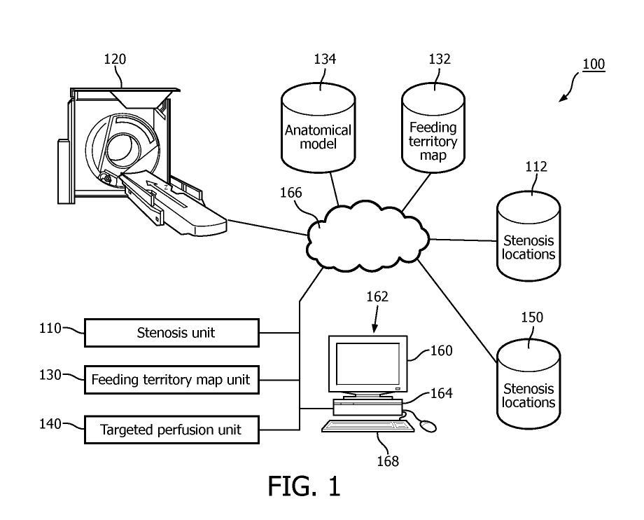

Initially referring to FIG. 1, a targeted perfusion protocol system 100 is schematically illustrated. A stenosis unit 110 identifies stenosis locations 112 in an organ of a patient, such as a heart. The stenosis is an abnormal narrowing in the arterial lumen, such as in the coronary artery of the heart. In one embodiment, the stenosis is identified from a prior volumetric image of the organ, e.g. prior imaging study. In one embodiment, the stenosis can include a stent location. In one embodiment, the stenosis unit 110 controls a scanning device 120, such as a CT scanner, an x-ray scanner, a magnetic resonance (MR) scanner, combinations and the like, to scan the heart according to an angiography protocol. The scanning device 120 according to the angiography protocol generates the location(s) of stenosis 112, such as in a volumetric image of the organ, using techniques known in the art. For example, a CT angiography (CTA) protocol scans the entire heart in a helical CT scan, which is a lower dose scan than a perfusion scan. The scan can include a contrast agent, which contrasts the arterial vessel. A coronary artery vessel tree is segmented. Diameters along the vessel tree can be measured and a fractional flow reserve (FFR) computed (FFR-CT). Based on the spatially located segmented arterial vessel, diameters, and FFR-CT, a location of stenosis is identified.

A feeding territory map unit 130 maps the location of the stenosis 112 to positions of a CT perfusion scan coverage using a feeding territory map 132. A feeding territory map associates locations or points along an arterial vessel tree with spatial locations of the organ tissue. The associated locations along the arterial vessel tree correspond to tissues fed by portions of arterial vessel tree downstream of arterial blood flow. For example, a branch along the coronary artery vessel tree is mapped to myocardial tissue supplied with arterial blood flow by the branch. The feeding territory map 132 can be initially constructed from a sampling of one or more healthy patient hearts scanned with a perfusion protocol, and associating branches with specific areas of myocardial tissue. In one embodiment, the feeding territory can be localized to a 2-3 centimeter (cm) axial or z coverage, or smaller depending on the size of the arterial branch. In one embodiment, the initially constructed map can be further refined or fit to a prior imaged organ of the patient using an anatomical model 134. The anatomical model 134 can deformably dimension the feeding territory map 132 using techniques known in the art, such as triangular mesh surfaces.

The feeding territory map unit 130 can map the locations of the stenosis 112 identified in the prior or generated image, e.g. a CTA image, using the dimensioned feeding territory map 132 or anatomically corresponding positions using a non-dimensioned feeding territory map 132 to portions of the organ tissues supplied downstream of the stenosis. In some instances, the CTA image is generated from a scan that uses one seventh or less ionizing radiation dose than a corresponding CT perfusion scan. The portions of the organ tissues supplied downstream of the stenosis are used to determine positional parameters of a targeted perfusion scan. For example, the feeding territory map 132 can include for each smallest branch of the arterial vessel tree, spatial locations of organ tissues, such as voxels corresponding to each smallest branch. The feeding territory map 132 can include an association of each smallest branch to larger segments and hierarchically to the whole arterial vessel tree. Thus, smallest branches in any branch with a stenosis can be identified hierarchically and in turn, the organ tissues, such as myocardium, identified.

With the dimensioned feeding territory map 132, the locations of the stenosis 112 correspond spatially with locations along the arterial vessel tree that associate with organ tissues. The organ tissues fed by downstream branches according to the dimensioned feeding territory map 132 also correspond spatially with tissues of the imaged organ of the patient used to spatially fit the dimensioned feeding territory map 132. The z positions of corresponding tissues of the patient are used as positional parameters for the targeted perfusion scan.

With the non-dimensioned feeding territory map 132, the locations of the stenosis 112 correspond to anatomical locations along the arterial vessel tree. The anatomical location of the stenosis along the arterial vessel tree, e.g. in the patient image, is identified that corresponds an anatomical location along the arterial vessel tree in the non-dimensioned feeding territory map 132. From the associated organ tissues fed in downstream branches, the anatomical locations of the associated organ tissues are determined and use to anatomically locate the corresponding organ tissues of the patient used as z positional parameters for the targeted perfusion scan.

A targeted perfusion unit 140 controls a CT scanner 120 to scan a portion of the patient organ using coverage determined from the feeding territory map 132. A scout and/or pilot scan can identify the organ location in the patient and positions of anatomical locations in the organ. The targeted perfusion unit 140 uses the positional information, such as anatomical positions in the scout scan along the z-axis of the scanner and corresponding positions determined from the feeding territory map 132 to position the patient in the field of view or coverage during uptake of a contrast agent during a perfusion scan. The feeding territory map 132 can provide a start position and an end position of the fed organ tissues. The field of view in a large coverage scanner, i.e. the coverage and hence, a dose, can be limited to the start and end positions. The field of view in a limited field of view scanner can be positioned to the start and end positions. The coverage can include a margin of error.

The targeted perfusion unit 140 generates a perfusion image of the scanned portion of the patient tissue. The scanned portion of the patient tissue is determined from the axial start and end positions of the fed tissues downstream of the stenosis based on the feeding territory map 132. The generated perfusion image can be stored in a storage subsystem 150, such as a Picture Archiving and Communication System (PACS), a Radiology Information System (RIS), Electronic Medical Record (EMR), and the like or displayed on a display device 160 of a computing device 162.

The stenosis unit 110, the feeding territory map unit 130, and the targeted perfusion unit 140 comprise one or more configured processors 164, e.g., a microprocessor, a central processing unit, a digital processor, and the like). The one or more configured processors 164 are configured to execute at least one computer readable instruction stored in a computer readable storage medium, which excludes transitory medium and includes physical memory and/or other non-transitory medium. The one or more processors 164 may also execute one or more computer readable instructions carried by a carrier wave, a signal or other transitory medium. The one or more processors 164 can include local memory and/or distributed memory. The one or more processors 164 can include hardware/software for wired and/or wireless communications over a network 166. For example, the lines indicate communications paths between the various components which can be wired or wireless. The one or more processors 164 can comprise the computing device 162, such as a desktop computer, a laptop computer, a body worn computing device, a smartphone, a tablet, and/or cooperative/distributed computing devices including one or more configured servers (not shown). The computing device 162 can include one or more input devices 168 which receive commands, such as identifying and/or confirming the locations of stenosis 112 in a volumetric image, displaying the perfusion scan generated image, displaying the feeding territory map 132, identifying coverage positions relative to the scout/pilot scan, confirming coverage, and the like.

The stenosis locations 112, the anatomical model 134, the feeding territory map 132 are represented as digital data sets stored on an electronic storage medium or computer memory. The digital data sets can include a Digital Imaging and Communications in Medicine (DICOM) format or other suitable image or volumetric format.

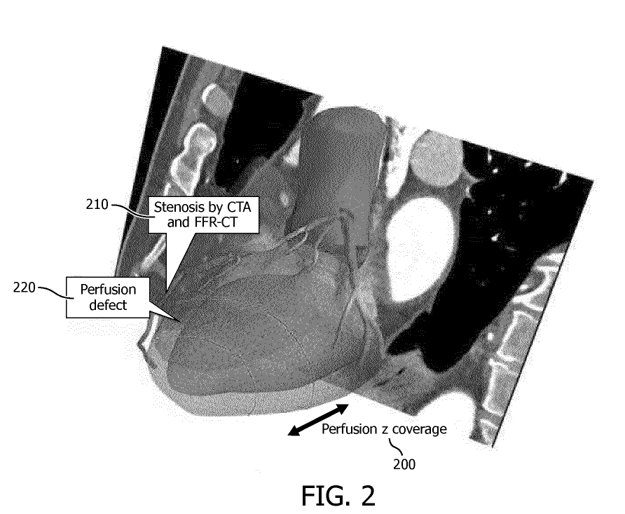

With reference to FIG. 2, an exemplary heart scan targeted coverage 200 with a stenosis 210 and a corresponding perfusion defect 220 is illustrated in a cut-away two dimensional perspective view of the heart and the coronary artery. The stenosis 210 is identified from the CTA image and FFR-CT computation. The perfusion defect 220 is analyzed according the targeted perfusion scan. The location of the perfusion defect 220 is determined from the feeding territory map 132 based on the location of the stenosis 210, e.g. tissues fed by arterial branches downstream of the stenosis 210.

The targeted coverage 200, or axial start and end positions in the targeted perfusion scan, are determined from the corresponding axial limits of fed tissues downstream from the stenosis 210. For example, a start position corresponds to a first z position of the fed territory. The end position correspond to a second z position of the fed territory and the first and second z positions limit the coverage of the scan with the fed territory disposed in between the first and second z positions. In one embodiment, the first and/or the second z positions can include a margin of error, such as an additional distance adjacent to the fed territory. In some instances the margin of error can be measured based on the sampling of healthy hearts used to construct the feeding territory map 132.

In some instances, the targeted coverage 200 reduces the dose received by the patient by reducing the coverage of tissues in a perfusion scan, e.g. less than the entire organ. The targeted coverage 200 during a targeted perfusion scan includes the organ defect and the monitoring and scanning of the tissue defects during uptake of the contrast agent.

In some instances, more than one stenosis 210 is identified for a targeted perfusion scan. In one embodiment, the target coverage 200 is expanded to include corresponding axial positions for all fed tissues. In one embodiment, multiple targeted coverages are identified within the limits of the CT scanner 120 constraints. For example, a first targeted coverage of a first start position z^{s}₁ and a first end position z^{e}₁ corresponds to a first fed territory, and a second targeted coverage of a second start position Z^{s}₂ and a second end position z^{e}₂ corresponds to a second fed territory. The first and second targeted coverage are within the field of view constraints of the CT scanner 120 and collimated in between. In another embodiment, the first and second targeted coverage are not within the field of view constraints of the CT scanner 120 and separate targeted perfusion scans are performed. In some instances, the locations of the fed tissues based on the feeding territory map 132 are used to prioritize the separate targeted perfusion scans.

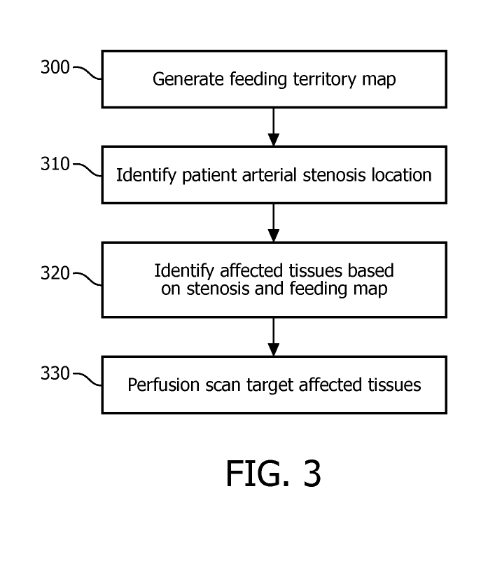

With reference to FIG. 3, an embodiment of a method of targeting perfusion scanning coverage is flowcharted. At 300, a feeding territory map 132 is generated based on a sampling of one or more healthy organs, e.g. according to perfusion studies. The feeding territory map 132 includes the arterial vessel and the corresponding spatially located tissues fed by each branch of the arterial vessel. For example, for each spatial location of an organ tissue represented by a voxel, values include a mapping to the arterial vessel branch and upstream branches that supply oxygenated blood.

A location of the stenosis 112 is identified at 310. The identification can include the generation of the CTA image and analysis using FFR-CT. The location of the stenosis 112 along the arterial vessel tree is identified, such as a branch along the coronary artery vessel tree. The location of the stenosis 112 can include a location of a stent or use other patient information to identify the location the stenosis 112.

At 320 the organ tissues fed downstream of the location of the stenosis 112 are determined from the feeding territory map 132. Axial positions of the determined organ tissues delineate the targeted coverage 200.

The targeted coverage 200 is input to the CT scanner 120 as parameters which control the coverage of the CT scanner 120 during a perfusion scan at 330. The perfusion scan scans the organ tissues within the targeted coverage 200 including during uptake of the contrast agent during the perfusion scan. A perfusion image is generated, which is a time dependent three dimensional image of the organ tissues scanned during the perfusion scan and can be displayed on the display device 160 and/or stored in the storage subsystem 150.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A system for a targeted perfusion scan, comprising:
a computed tomography (CT) scanner configured to perform a perfusion scan of a portion of tissues of an organ;
a feeding territory map configured to map arterial locations of an arterial vessel tree to spatially located organ tissues of the organ fed by the arterial locations; and
a targeted perfusion unit including one or more processors configured to determine targeted coverage from a location of a stenosis and the feeding territory map, and to control the CT scanner to perform the perfusion scan according to the determined targeted coverage.

2. The system according to claim 1, further including:
a feeding territory map unit including the one or more processors configured to generate the feeding territory map based on a sampling of one or more healthy organ arterial vessel trees and corresponding organ tissues.

3. The system according to claim 1, further including:
a stenosis unit including the one or more processors configured to determine the location of the stenosis from a computed tomography angiography scan of the arterial vessel tree.

4. The system according to claim 1, wherein the targeted perfusion unit is further configured to control the computed tomography (CT) scanner to limit scanning to the targeted coverage during the perfusion scan.

5. The system according to claim 1, wherein the targeted coverage includes positions in between a first axial position and a second axial position of the organ within a field of view of the CT scanner.

6. The system according to claim 1, wherein the targeted coverage includes a distance between axial positions less than a field of view of the CT scanner.

7. The system according to claim 1, wherein the feeding territory map includes a mapping from each end branch of the arterial vessel tree hierarchically to connected upstream branches and mapping to the organ tissues feed by each branch of the arterial vessel tree.

8. The system according to claim 1, wherein the feeding territory map maps heart myocardial tissues fed by the coronary artery vessel tree.

9. The system according to claim 2, wherein the feeding territory map unit fits the feeding territory map to the locations of the stenosis using an anatomical model of the organ and arterial vessel tree.

10. A method of targeting a perfusion scan, comprising:
determining targeted coverage from a location of a stenosis and a feeding territory map; and
controlling a computed tomography (CT) scanner scan a portion of organ tissues according to the targeted coverage during the perfusion scan.

11. The method according to claim 10, further including:
generating the feeding territory map based on a sampling of one or more healthy organ arterial vessel trees and corresponding organ tissues.

12. The method according to claim 10, further including:
determining the location of the stenosis from a computed tomography angiography scan of the arterial vessel tree.

13. The method according to claim 10, wherein controlling includes:
displaying a CT perfusion image generated by the perfusion scan on a display device.

14. The method according to claim 10, wherein the targeted coverage includes positions in between a first axial position and a second axial position of the organ within a field of view of the CT scanner.

15. The method according to claim 10, wherein the targeted coverage includes a distance between axial positions less than a field of view of the CT scanner.

16. The method according to claim 10, wherein the feeding territory map includes a mapping from smallest branches of the arterial vessel tree hierarchically to larger branches and mapping to the organ tissues feed by each branch of the arterial vessel tree.

17. The method according to claim 10, wherein the feeding territory map maps heart myocardial tissues fed by the coronary artery vessel tree.

18. The method according to claim 10, wherein the feeding territory map unit fits the feeding territory map to the locations of the stenosis using an anatomical model of the organ and arterial vessel tree.

19. A non-transitory computer readable storage medium containing instructions that when executed by one or more processors:
determine a targeted coverage of a computed tomography (CT) scanner from a location of a stenosis in an arterial vessel tree of an organ and a feeding territory map that maps arterial locations of the arterial vessel tree to spatially located organ tissues of the organ fed by the arterial locations; and
control the CT scanner to perform a targeted CT perfusion scan according to the targeted coverage.

20. The non-transitory computer readable storage medium containing instructions that when executed by one or more processors according to claim 19, further include:
control the CT scanner to perform a CT angiography scan that generates the volumetric image of the organ; and
identify the location of the stenosis in the arterial vessel tree of the organ from the volumetric image of the organ.
